(19) United States
(12) Patent Application Publication
(10) Pub. No.: US 2021/0137531 A1
(43) Pub. Date: May 13, 2021

(54) INTEGRATED TOURNIQUET SYSTEM

(71) Applicant: Javier Rivero, Miami Beach, FL (US)

(72) Inventor: Javier Rivero, Miami Beach, FL (US)

(21) Appl. No.: 15/645,114

(22) Filed: Jul. 10, 2017

(51) Int. Cl. A61B 17/135; B63C 9/08; B63C 9/105; A41D 13/012; A41D 7/00; A61B 17/132; A61B 90/50; A41D 13/00

(52) U.S. Cl. CPC A61B 17/135; B63C 9/081; B63C 9/1055; A41D 13/0012; A41D 7/005; A61B 17/1322; A61B 90/50; A41D 13/0125

(57) ABSTRACT

An integrated tourniquet system is provided that, in some embodiments, provides for a specially designed pocket integrated within a garment or flotation device wherein the pocket is configured to removably retain a one-handed tourniquet having, in some embodiments, a windlass system.

### CROSS-REFERENCE TO RELATED APPLICATION

Not applicable.

### TECHNICAL FIELD

The present invention relates generally to the field of clothing and more specifically relates to an integrated tourniquet system.

### BACKGROUND

Tourniquets are an effective means of arresting life-threatening external hemorrhage from a limb injury. Life threatening limb hemorrhage not controlled by simple methods can turn deadly in seconds. A boating accident or capsizing can lead to major extremity trauma. Also, the potential for a shark attack while floating in the ocean waiting for a rescue watercraft is high. If there is an encounter with a boating accident trauma or marine life, a quick first aid remedy while floating such as a tourniquet is very important. Therefore, tourniquets are crucially important items.

Certain commercial tourniquets have been proven in studies to be more effective than using an improvised device that has been demonstrated to take more time to apply or may lead to an increased risk of complications. For example, the Combat Application Tourniquet® made by North American Rescue Products, Inc., for operational use showed 100% effectiveness in occluding distal arterial flow using human volunteers in experimental studies. There are numerous situations when tourniquet application can be necessary and lifesaving in the civilian pre-hospital setting. Tourniquets are no longer only considered as “last resort” device. Many people are familiarizing themselves with this simple piece of equipment and becoming prepared to use it in appropriate cases without an irrational fear of complications.

Many shark attacks/marine type traumatic incidents occur worldwide, each year. A surfer/swimmer armed with a tourniquet on his/her person can quickly apply it prior to making it back to shore, completely preventing severe hemorrhage. Waiting for a swimmer/surfer to be brought onto shore in order to start first aid is a waste of critical minutes, especially when a major artery is compromised. The femoral artery is a considerably large artery and bleeds profusely when severed. Often times, the individual loses consciousness and dies within minutes. In fact, recently a tourniquet has saved two Hawaiian surfers' lives when their limbs were torn away by sharks in past attacks. What is needed therefore is a swimwear line, specifically board shorts for men, that have an integrated tourniquet.

Various attempts to solve this problem have been made. One such attempt can be seen with respect to U.S. Patent Application No. 2008/0221612, incorporated by reference in its entirety herein, which generally discloses a garment having two integrated tourniquets for each limb. While this disclosure does provide for multiple integrated tourniquets, the outdated tourniquets provided are of little use compared with today's advanced models, such as for example, a Combat Application Tourniquet®.

Another attempt can be seen with respect to U.S. Pat. No. 8,900,266, incorporated by reference in its entirety herein, which generally discloses an advanced tourniquet. While this device does appear to work quickly and easily, it fails to provide for integration within a garment or a flotation device.

Yet another attempt can be seen with respect to U.S. Pat. No. 7,981,135, incorporated by reference in its entirety herein, which generally discloses a garment having an integrated tourniquet. While this disclosure does provide for an easy to use embedded tourniquet, and a design that appears fashion-forward, it fails to provide for the advanced tourniquet mentioned above.

Various attempts have been made to solve the problems which may be found in the related art but have thus far been unsuccessful. A need exists for an advanced integrated tourniquet system embedded within either a garment or a life saving device.

### SUMMARY OF THE INVENTION

It is to be understood that in the present disclosure, all embodiments are provided as illustrative and non-limiting representatives of many possible embodiments. In addition, the terms “is,” “can,” “will,” and the like are herein used as synonyms for and interchangeable with terms such as “may,” “may provide for,” and “it is contemplated that the present invention may” and so forth.

The present invention relates to an advanced integrated tourniquet system that overcomes the deficiencies of the prior art. It is contemplated that the present invention may provide an advanced integrated tourniquet system that can work in either a garment or a life saving device.

For purposes of summarizing, certain aspects, advantages, and novel features of the present invention are provided herein. It is to be understood that not all such aspects, advantages, or novel features may be provided in any one particular embodiment. Thus, the disclosed subject matter may be embodied or carried out in a manner that achieves or optimizes one aspect, advantage, or novel feature or group of features without achieving all aspects, advantages, or novel features as may be taught or suggested.

In view of the foregoing disadvantages inherent in the known art, the present invention provides a novel an advanced integrated tourniquet system. The general purpose of the present invention, which will be described subsequently in greater detail, is to provide an advanced integrated tourniquet system that can save lives in nature, particularly in open water. The features of the invention which are believed to be novel are particularly pointed out and distinctly claimed in the concluding portion of the specification. These and other features, aspects, and advantages of the present invention will become better understood with reference to the following drawings and detailed description.

In one embodiment, the present invention may provide for an advanced integrated tourniquet system that is operable to integrate with either a garment or a flotation device, such as a personal flotation device (PFD), a life preserver, a life vest, a life belt, a life jacket, or a flotation toy, such as a blow-up innertube or arm-floaties. In some embodiments, the present invention may be configured to be operative in items designed for open water. It is contemplated that in some embodiments the present invention may incorporate a life-saving tourniquet into a clothing line.

In some embodiments, the present invention may provide for a discreet pocket inside the lining of a garment such as a pair of board shorts, wherein the compartment may be specially configured to store a tourniquet similar to the CAT (Combat Application Tourniquet®), in that the intended tourniquet is contemplated to, in some embodiments, have one-handed applicability, a windlass system, a tightening-by-cylinder-and-chord method of tightening, and so forth. The present invention is contemplated to place the life-saving tourniquet tool conveniently in a pocket, wherein the pocket is that is strategically located and uniquely configured to receive, retain, and release the tourniquet as needed, providing accessibility while at the same time not affecting or limiting a user's athletic or active performance. In some embodiments, an epi-pen can also fit/be placed inside the present invention's pocket as an alternative life saving device.

The unique features of this product provide the following benefits for consumers everywhere: it may reduce the extent of injury, disability, and death associated with water sports, dangerous training routines, and recreation; it may allow any outdoor sporting enthusiast to be able to quickly save his/her own life as well as any person they may be with or encounter with a life threating hemorrhaging type of injury; it may enable self-preservation and buddy preservation; it may be convenient and good for on-the-go activities involving lots of movement; it may set the safety standard for every life-saving PFD (life jacket).

### BRIEF DESCRIPTION OF THE DRAWINGS

These and other features, aspects and advantages of the present invention will become better understood with regard to the following description, appended claims, and accompanying figures where:

FIG. 1 shows a rear perspective view of an integrated tourniquet system in accordance with one embodiment of the invention.

FIG. 2 shows a rear perspective view of an integrated tourniquet system in accordance with one embodiment of the invention.

FIG. 3 shows a rear perspective view of an integrated tourniquet system in accordance with one embodiment of the invention.

FIG. 4 shows a top perspective view of an integrated tourniquet system in accordance with one embodiment of the invention.

FIG. 5 shows a top perspective view of an integrated tourniquet system in accordance with one embodiment of the invention.

### DETAILED DESCRIPTION

The present invention overcomes the limitations of the prior art by providing a new and more effective integrated tourniquet system.

All dimensions specified in this disclosure are by way of example only and are not intended to be limiting. Further, the proportions shown in these Figures are not necessarily to scale. As will be understood by those with skill in the art with reference to this disclosure, the actual dimensions and proportions of any embodiment or element of an embodiment disclosed in this disclosure will be determined by its intended use.

It is to be understood that the drawings and the associated descriptions are provided to illustrate potential embodiments of the invention and not to limit the scope of the invention. Reference in the specification to “one embodiment” or “an embodiment” is intended to indicate that a particular feature, structure, or characteristic described in connection with the embodiment is included in at least an embodiment of the invention. The appearances of the phrase “in one embodiment” or “an embodiment” in various places in the specification are not necessarily all referring to the same embodiment.

Throughout the drawings, reference numbers are re-used to indicate correspondence between referenced elements. In addition, the first digit of each reference number indicates the figure where the element first appears.

As used in this disclosure, except where the context requires otherwise, the term “comprise” and variations of the term, such as “comprising”, “comprises” and “comprised” are not intended to exclude other additives, components, integers or steps.

In the following description, specific details are given to provide a thorough understanding of the embodiments. However, it will be understood by one of ordinary skill in the art that the embodiments may be practiced without these specific detail. Well known features, elements or techniques may not be shown in detail in order not to obscure the embodiments.

Turning attention to FIG. 1, a rear perspective view of a set of an integrated tourniquet system in accordance with one embodiment of the invention is shown. In the embodiment depicted, a viewer may perceive flap 101 and shorts 102.

With respect to FIG. 2, a rear perspective view of a set of an integrated tourniquet system in accordance with one embodiment of the invention is shown. In the embodiment depicted, a viewer may perceive hook and loop fastening element 201 and pouch 202 (which may be elastomer-nylon material), as well as flap 101 and shorts 102.

With respect to FIG. 3, a rear perspective view of a set of an integrated tourniquet system in accordance with one embodiment of the invention is shown. In the embodiment depicted, a viewer may perceive tourniquet 301, as well as flap 101 and shorts 102.

With respect to FIG. 4, a top perspective view of a set of an integrated tourniquet system in accordance with one embodiment of the invention is shown. In the embodiment depicted, a viewer may perceive solid gel molding 401, as well as flap 101 and shorts 102.

With respect to FIG. 5, a top perspective view of a set of an integrated tourniquet system in accordance with one embodiment of the invention is shown. In the embodiment depicted, a viewer may perceive embedded gel lining 501, as well as solid gel molding 401, flap 101 and shorts 102.

### CONCLUSIONS, RAMIFICATIONS, AND SCOPE

Although the present invention has been described with a degree of particularity, it is understood that the present disclosure has been made by way of example and that other versions are possible. As various changes could be made in the above description without departing from the scope of the invention, it is intended that all matter contained in the above description or shown in the accompanying drawings shall be illustrative and not used in a limiting sense. The spirit and scope of the appended claims should not be limited to the description of the preferred versions contained in this disclosure.

All features disclosed in the specification, including the claims, abstracts, and drawings, and all the steps in any method or process disclosed, may be combined in any combination, except combinations where at least some of such features and/or steps are mutually exclusive. Each feature disclosed in the specification, including the claims, abstract, and drawings, can be replaced by alternative features serving the same, equivalent or similar purpose, unless expressly stated otherwise. Thus, unless expressly stated otherwise, each feature disclosed is one example only of a generic series of equivalent or similar features.

Any element in a claim that does not explicitly state “means” for performing a specified function or “step” for performing a specified function should not be interpreted as a “means” or “step” clause as specified in 35 U.S.C. § 112.

While the integrated tourniquet system generally described herein has been disclosed in connection with a number of embodiments shown and described in detail, various modifications should be readily apparent to those of skill in the art.

What is claimed is:

1. A tourniquet system comprising:
a garment;
a one-handed tourniquet; and
a pocket within the garment, wherein the pocket is configured to removably receive the tourniquet.

2. The system of claim 1, wherein the tourniquet is configured for use in water.

3. The system of claim 1, wherein the garment is a pair of board shorts.

4. The device of claim 3, further comprising solid gel molding on the inside of the garment.

5. The device of claim 4, further comprising a lining, wherein the lining is configured to fixedly connect the solid gel molding to the garment.

6. The device of claim 5, wherein the lining is an embedded gel lining.

7. A tourniquet system, comprising:
a flotation device;
a one-handed tourniquet; and
a pocket within the life saving device, wherein the pocket is configured to removably receive the tourniquet.

8. The system of claim 8, wherein the life saving device is a life jacket.

9. The system of claim 8, wherein the life saving device is a life vest.

10. The system of claim 8, wherein the life saving device is a life preserver.

11. The system of claim 8, wherein the life saving device is a flotation toy.
